(19) United States
(12) Patent Application Publication
(10) Pub. No.: US 2006/0251824 A1
(43) Pub. Date: Nov. 9, 2006

(54) Coating of medical devices with solids

(76) Inventors: Dennis R. Boulais, Danielson, CT (US); Mary Jo Timm, Littleton, MA (US)

Correspondence Address: COOLEY GODWARD LLP;ATTN: PATENT GROUP, THE BOWEN BUILDING, 875 15TH STREET, N.W. SUITE 800, WASHINGTON, DC, 20005-2221, US

(21) Appl. No.: 11/121,926

(22) Filed: May 5, 2005

(51) Int. Cl. A61L 33/00; B05D 1/04

(52) U.S. Cl. 427/458; 427/2.1

(57) ABSTRACT

The present invention satisfies this and other needs by providing methods for coating medical devices with solid agents. The coating processes described in the present invention may be self-limiting in the total amount of solid agent deposited on the surface of the medical device and hence may provide medical devices with substantially uniform coating thickness.

### TECHNICAL FIELD

The present invention relates to coated medical devices. More particularly, the present invention relates to medical devices coated with solid agents such as powders.

### BACKGROUND

Medical device surfaces are often coated with various agents that have useful properties, for example, which may aid in treatment of a localized disease (e.g., heart disease, occluded body lumens, etc.). Such coated medical devices are often more effective than systemic drug administration in delivering significant concentrations of therapeutic agents to the necessary location in an organism. Further, drug release coatings on medical devices may provide for controlled release, including sustained release, of therapeutic agents. Medical devices may also be coated with materials such as radiopaque materials, radioactive materials, coatings which enhance lubriciousness, hydrophilic coatings, coatings which increase biocompatibility, etc.

Typically, coatings are applied to medical devices by conventional processes such as dipping, spraying, plasma polymerization, wiping, pad printing, etc. However, since some solid agents cannot be dissolved in solvents, the above methods cannot be used to coat medical devices with these insoluble agents. Currently, medical devices are coated with solid agents by sprinkling a powder of the solid agent on the surface of the medical device or by rolling the medical device in a powder of the solid agent. However, these coating methods often fail to provide necessary uniform coating thickness on both individual medical devices and batches of medical devices, which may lead to inferior performance.

Accordingly, what is needed are new methods for coating medical devices with solid agents. Preferably, these methods will provide medical devices with substantially uniform coating thickness.

### SUMMARY

The present invention satisfies this and other needs by providing methods for coating medical devices with solid agents. The coating processes described in the present invention may be self-limiting in the total amount of solid agent deposited on the surface of the medical device and hence may provide medical devices with substantially uniform coating thickness.

### DETAILED DESCRIPTION

Reference will now be made in detail to preferred embodiments of the invention. While the invention will be described in conjunction with particular embodiments, it will be understood that it is not intended to limit the invention to those specific embodiments. Numerous specific details are set forth to provide a thorough understanding of the present invention. Accordingly, the skilled artisan will appreciate that the present invention may be practiced without some or all of these specific details and includes alternatives, modifications and equivalents within the scope of the invention as defined by the appended claims.

The present invention provides methods for coating medical devices with solid agents. The coating processes described herein may be self-limiting in the total amount of solid agent deposited on the surface of the medical device and hence may provide medical devices with substantially uniform coating thickness and minimal defects. Further, the methods of the present invention may be used to coat medical devices which are not uniformly coated by use of conventional technologies.

### Solid Agents

In some embodiments, a solid agent is applied to a surface of a medical device by electrodeposition. Typically, the solid agent will be electrically charged (i.e., contain electrically charged functional groups such as carboxylic acids, amines, etc.) and will be in powder form. In one embodiment, the particle size of the solid agent is substantially uniform.

In other embodiments, a powder blend of a solid agent and an electrically chargeable filler is applied to a surface of a medical device by electrodeposition. The solid agent may be either electrically neutral or electrically charged. The electrically chargeable filler may be inert or may have biological or therapeutic activity. Preferably, the powder blend is an mixture of the solid agent and an electrically chargeable filler and may be made by methods known in the art. In one embodiment, the particle size of the powder blend is substantially uniform.

Electrodeposition of solid agents and powder blends of solid agent and electrically chargeable filler may be accomplished using methods described in the art. For example, methods described in U.S. Pat. Nos. 6,372,246, 4,197,289, 5,714,007 and 5,695,826 may be used for electrodeposition of solid agents and powder blends of solid agent and electrically chargeable filler on the surface of medical devices.

The electrodeposition process may be self limiting in the total amount of solid agent or powder blend of solid agent and electrically charged filler deposited on a surface of a medical device. The attraction of charged solid agent or powder blend of solid agent and electrically charged filler to a surface of a medical device will decrease as the amount of charged material deposited on the surface increases, thus self limiting the amount of material which can be deposited on the surface of the medical device.

Materials for the electrically chargeable filler can be a variety of agents provided that requirements for biological and hematological compatibility are satisfied. Accordingly, the electrically chargeable filler can be, for example, therapeutic agents, radiopaque materials, radioactive materials, polymeric materials, sugars, waxes and fats as described, herein.

The solid agents used in the present invention are any desirable suitable solid agents. Suitable solid agents include any solid form of a compound or material or mixture of compounds or materials. Specifically included as solid agents are powders or granular materials of any sort. In one embodiment, the solid agent is insoluble in any solvent or solvent mixture. Exemplary solid agents include, but are not limited to, therapeutic agents (as defined below), radiopaque materials (e.g., iodine, iodine salts, iodine compounds, barium, barium salts, barium compounds, tungsten, rhenium, osmium, palladium, platinum, gold silver, tantalum, iridium, alloys thereof, combinations thereof etc.), radioactive materials, polymeric materials, sugars, fats, etc. as described herein and combinations thereof.

In one embodiment, the solid agent is a therapeutic agent. In another embodiment, the solid agent is a mixture of two or more therapeutic agents. In still another embodiment, the solid agent is a mixture of two or more therapeutic agents, where at least one of the therapeutic agents is electrically charged.

### Adhesive Agents

In still other embodiments, the surface of a medical device may be first coated with an adhesive agent, which may be either a liquid or solid The adhesive agent may be inert or may be biologically or therapeutically active. Any method known in the art (e.g., ion beam assisted deposition, ion beam, ion beam implantation, air suspension as described in U.S. Pat. No. 6,368,658, the method described in U.S. Pat. No. 6,322,847, dipping, spraying, brushing, wiping, pad printing, electrostatic liquid spraying, electrostatic powder coating, etc.) may be used to coat the surface of the medical device with the adhesive agent. A solid agent can then be applied to the surface of the medical device, which has been coated with the adhesive agent. Alternatively, a powder blend of a solid agent and a filler may be applied to the surface of the medical device. The filler may be inert or may have biological or therapeutic activity. Preferably, the powder blend is an intimate mixture of solid agent and filler and may be made by methods known the skilled artisan. In one embodiment, the particle size of the powder blend is substantially uniform.

As used herein the term “adhesive agent” means an agent that is tacky or sticky such that the adhesive agent allows two items to be coupled, at least temporarily, together. For example, adhesive agents include, but are not limited to waxes and paraffin.

Materials for the filler can be a variety of agents provided that requirements for biological and hematological compatibility are satisfied. Accordingly, the filler can be, for example, therapeutic agents, radiopaque materials, radioactive materials, polymeric materials, sugars, waxes and fats as described, infra.

### Methods

Conventional methods known to those of skill in the art may be used to apply the solid agent or powder blend to the surface of the medical device. For example, methods described in U.S. Pat. Nos. 4,830,279, 4,526,804 and 5,769,276 may be used to practice the current invention. The adhesive coating process described herein may be self limiting in the total amount of solid agent or powder blend of solid agent and filler deposited on a surface of a medical device since material which failed to contact the adhesive agent would fail to adhere to the surface of the medical device.

It should be noted that in the processes described herein (i.e., electrodeposition and adhesive coating) the amount of solid agent deposited on the surface of a medical device may be readily by changing the ratio of solid agent to filler in the powder blend. In one embodiment, the ratio of solid agent to filler is between about 1:10 to about 10:1. In another embodiment, the ratio of solid agent to filler is between about 1:5 to about 5:1. In still another embodiment, the ratio of solid agent to filler is between about 1:2 to about 2:1. In still another embodiment, the ratio of solid agent to filler is about 1:1.

Another method of adjusting the amount of solid agent deposited on a surface of a medical device in the processes described herein is by controlling the particle size of the solid agent or the powder blend of solid agent and filler. For example, the particle size of the solid agent or the powder blend of the solid agent and filler could be tailored to fit the geometry of the surface of the medical device. Thus, medical devices with physically small features could be coated with a solid agent or a powder blend of solid agent and filler of small particle size. This would allow for more uniform coating of a medical device surface than coating with particles relatively large to the medical device surface. Accordingly, those of skill in the art will appreciate that the particle size of the solid agent or powder blend of solid agent and filler will vary with the size of the surface of the medical device.

In one embodiment, the particle size of a solid agent varies between about 1 μm and about 500 μm. In another embodiment, the particle size of a solid agent varies between about 5 μm and about 100 μm. In still another embodiment, the particle size of a solid agent varies between about 10 μm and about 50 μm.

In one embodiment, the particle size of a powder blend of solid agent and filler varies between 1 μm and about 500 μm. In another embodiment, the particle size of a powder blend of solid agent and filler varies between 5 μm and about 100 μm. In still another embodiment, a particle size of the powder blend of solid agent and filler varies between 10 μm and about 50 μm.

### Coatings

Agents used to coat medical devices include, for example, therapeutic agents radiopaque materials (e.g., iodine, its salts and compounds, barium, its salts and compounds, tungsten, rhenium, osmium, palladium, platinum, gold silver, tantalum, iridium, alloys thereof, etc.), radioactive materials, polymeric materials, sugars, waxes and fats. Any of the agents above may be used alone or in any combination with other of the above agents with at least one of the agents being a solid agent or a powder blend of a solid agent and filler. When used in combination, any of the above agents may be applied simultaneously, after or before the initially applied agent. For example, when used with therapeutic agents, any of above agents may be applied simultaneously, after or before the therapeutic agent.

As used herein, the term “therapeutic agent” includes, but is not limited to, any therapeutic, such as drugs and includes genetic materials and biological materials. Suitable genetic materials include DNA or RNA, such as, without limitation, DNA/RNA encoding a useful protein and DNA/RNA intended to be inserted into a human body including viral vectors and non-viral vectors. Suitable viral vectors include, for example, adenoviruses, gutted adenoviruses, adeno-associated virus, retroviruses, alpha viruses (Semliki Forest, Sindbis, etc.), lentiviruses, herpes simplex virus, ex vivo modified cells (e.g., stem cells, fibroblasts, myoblasts, satellite cells, pericytes, cardiomyocytes, skeletal myocytes, macrophage), replication competent viruses (e.g., ONYX-015) and hybrid vectors. Suitable non-viral vectors include, for example, artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD).

Suitable biological materials include, for example, cells, yeasts, bacteria, proteins, peptides, cytokines, and hormones. Examples of suitable peptides and proteins include growth factors (e.g., FGF, FGF-1, FGF-2, VEGF, Endotherial Mitogenic Growth Factors, and epidermal growth factors, transforming growth factor α and β, platelet derived endothelial growth factor, platelet derived growth factor, tumor necrosis factor α, hepatocyte growth factor and insulin like growth factor), transcription factors, proteinkinases, CD inhibitors, thymidine kinase, and bone morphogenic proteins (BMPs), such as BMP-2, BMP-3, BMP-4, BMP-5, BMP-6 (Vgr-1), BMP-7 (OP-1), BMP-8. BMP-9, BMP-10, BMP-11, BMP-12, BMP-13, BMP-14, BMP-15, and BMP-16. Currently preferred BMPs are BMP-2, BMP-3, BMP-4, BMP-5, BMP-6, and BMP-7. These dimeric proteins can be provided as homodimers, heterodimers, or combinations thereof, alone or together with other molecules. The above materials are either commercially available and/or recombinantly produced or isolated. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include, for example, whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, macrophage, and satellite cells.

The term “therapeutic agent” also includes, but is not limited to, anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid, amlodipine and doxazosin; anti-inflammatory agents such as glucocorticoids, betamethasone, dexamethasone, prednisolone, corticosterone, budesonide, estrogen, sulfasalazine, and mesalamine; antineoplastic/antiproliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, taxol and its analogs or derivatives; anesthetic agents such as lidocaine, bupivacaine, and ropivacaine; anti-coagulants such as D-Phe-Pro-Arg chloromethyl ketone, an RGD peptide-containing compound, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick anti-platelet peptides; vascular cell growth promotors such as growth factors, Vascular Endothelial Growth Factors (VEGF, all types including VEGF-2), growth factor receptors, transcriptional activators, and translational promotors; vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents, vasodilating agents, and agents which interfere with endogenous vasoactive mechanisms; anti-oxidants, such as probucol; antibiotic agents, such as penicillin, cefoxitin, oxacillin, tobramycin; angiogenic substances, such as acidic and basic fibrobrast growth factors, estrogen including estradiol (E2), estriol (E3) and 17-beta estradiol; and drugs for heart failure, such as digoxin, beta-blockers, angiotensin-converting enzyme (ACE) inhibitors including captopril and enalopril.

Additional therapeutic agents include, but are not limited to, anti-proliferative drugs such as steroids, vitamins, and restenosis-inhibiting agents such as cladribine. Preferred restinosis-inhibiting agents include microtubule stabilizing agents such as Taxol, paclitaxel, paclitaxel analogues, derivatives, and mixtures thereof. For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl)glutamine, and 2′-O-ester with N-(dimethylaminoethyl)glutamide hydrochloride salt. Other preferred therapeutic agents include nitroglycerin, nitrous oxides, antibiotics, aspirins, digitalis and glycosides. In one embodiment, the therapeutic agent is sicolimus, dexamethasone, estradiol, tacrolimus, evercolimus, nitric oxide, mycophenloc acid and trapidil.

Examples of polymeric materials which may be used in the coating compositions of the present invention include, but not limited to, polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate, styrene-isobutylene copolymers and blends and copolymers thereof.

Other examples of polymers which may be used in the coating compositions of the present invention include polyurethane (BAYHDROL, etc.) fibrin, collagen and derivatives thereof, polysaccharides such as celluloses, starches, dextrans, alginates and derivatives, hyaluronic acid and squalene. Further examples of polymeric materials which may be used in the coating composition of the present invention include polymers that can be dissolved and cured or polymerized on the medical device or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyl resins, polycarbonates, polyoxymethylenes, polyimides, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (ethylene-propylene-diene) rubbers, fluorosilicones, polyethylene glycol, polysaccharides, phospholipids, and combinations thereof.

In one embodiment, the polymer is polyacrylic acid, as described in U.S. Pat. No. 5,091,205. In another embodiment, the polymer is a copolymer of polylactic acid and polycaprolactone. In yet another embodiment, the polymer is a polyester amide.

Preferably, for medical devices which undergo mechanical challenges, (e.g. expansion and contraction), the polymeric materials are selected from elastomeric polymers such as silicones (e.g., polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition adheres better to the surface of the medical device when the device is subjected to forces, stress or mechanical challenge.

Examples of hydrophobic polymers which may be used as polymeric coatings in the present invention include, but not are limited to, polyolefins, such as polyethylene, polypropylene, poly(1-butene), poly(2-butene), poly(1-pentene), poly(2-pentene), poly(3-methyl-1-pentene), poly(4-methyl-1-pentene), poly(isoprene), poly(4-methyl-1-pentene), ethylene-propylene copolymers, ethylene-propylene-hexadiene copolymers, ethylene-vinyl acetate copolymers, blends of two or more polyolefins and random and block copolymers prepared from two or more different unsaturated monomers; styrene polymers, such as poly(styrene), poly(2-methylstyrene), styrene-acrylonitrile copolymers having less than about 20 mole-percent acrylonitrile, and styrene-2,2,3,3,-tetrafluoropropyl methacrylate copolymers; halogenated hydrocarbon polymers, such as poly(chlorotrifluoroethylene), chlorotrifluoroethylene-tetrafluoroethylene copolymers, poly(hexafluoropropylene), poly(tetrafluoroethylene), tetrafluoroethylene, tetrafluoroethylene-ethylene copolymers, poly(trifluoroethylene), poly(vinyl fluoride), and poly(vinylidene fluoride); vinyl polymers, such as poly(vinyl butyrate), poly(vinyl decanoate), poly(vinyl dodecanoate), poly(vinyl hexadecanoate), poly(vinyl hexanoate), poly(vinyl propionate), poly(vinyl octanoate), poly(heptafluoroisopropoxyethylene), poly(heptafluoroisopropoxypropylene), and poly(methacrylonitrile); acrylic polymers, such as poly(n-butyl acetate), poly(ethyl acrylate), poly(1-chlorodifluoromethyl)tetrafluoroethyl acrylate, poly di(chlorofluoromethyl)fluoromethyl acrylate, poly(1,1-dihydroheptafluorobutyl acrylate), poly(1,1-dihydropentafluoroisopropyl acrylate), poly(1,1-dihydropentadecafluorooctyl acrylate), poly(heptafluoroisopropyl acrylate), poly 5-(heptafluoroisopropoxy)pentyl acrylate, poly 11-(heptafluoroisopropoxy)undecyl acrylate, poly 2-(heptafluoropropoxy)ethyl acrylate, and poly(nonafluoroisobutyl acrylate); methacrylic polymers, such as poly(benzyl methacrylate), poly(n-butyl methacrylate), poly(isobutyl methacrylate), poly(t-butyl methacrylate), poly(t-butylaminoethyl methacrylate), poly(dodecyl methacrylate), poly(ethyl methacrylate), poly(2-ethylhexyl methacrylate), poly(n-hexyl methacrylate), poly(phenyl methacrylate), poly(n-propyl methacrylate), poly(octadecyl methacrylate), poly(1,1-dihydropentadecafluorooctyl methacrylate), poly(heptafluoroisopropyl methacrylate), poly(heptadecafluorooctyl methacrylate), poly(1-hydrotetrafluoroethyl methacrylate), poly(1,1-dihydrotetrafluoropropyl methacrylate), poly(1-hydrohexafluoroisopropyl methacrylate), and poly(t-nonafluorobutyl methacrylate); polyesters, such a poly(ethylene terephthalate) and poly(butylene terephthalate); condensation type polymers such as and polyurethanes and siloxane-urethane copolymers; polyorganosiloxanes, i.e., polymeric materials characterized by repeating siloxane groups, represented by Ra SiO 4-a/2, where R is a monovalent substituted or unsubstituted hydrocarbon radical and the value of a is 1 or 2; and naturally occurring hydrophobic polymers such as rubber.

Examples of hydrophilic monomers which may be added to polymers include, but are not limited to, (meth)acrylic acid, or alkaline metal or ammonium salts thereof; (meth)acrylamide; (meth)acrylonitrile; those polymers to which unsaturated dibasic, such as maleic acid and fumaric acid or half esters of these unsaturated dibasic acids, or alkaline metal or ammonium salts of these dibasic adds or half esters, are added; those polymers to which unsaturated sulfonic, such as 2-acrylamido-2-methylpropanesulfonic, 2-(meth)acryloylethanesulfonic acid, or alkaline metal or ammonium salts thereof, are added; and 2-hydroxyethyl(meth)acrylate and 2-hydroxypropyl(meth)acrylate. Polyvinyl alcohol is also an example of hydrophilic polymer. Polyvinyl alcohol may contain a plurality of hydrophilic groups such as hydroxyl, amido, carboxyl, amino, ammonium or sulfonyl (—SO3). Hydrophilic polymers also include, but are not limited to, starch, polysaccharides and related cellulosic polymers; polyalkylene glycols and oxides such as the polyethylene oxides; polymerized ethylenically unsaturated carboxylic acids such as acrylic, methacrylic and maleic acids and partial esters derived from these acids and polyhydric alcohols such as the alkylene glycols; homopolymers and copolymers derived from acrylamide; and homopolymers and copolymers of vinylpyrrolidone.

Polymeric materials may be employed as primer layers for enhancing subsequent coating applications layers to control release of therapeutic agents (e.g., barrier diffusion polymers such as hydrophobic polymers for sustained release, thermal responsive polymers, pH responsive polymers, etc.), protective layers for underlying drug layers, biodegradable layers, biocompatible layers (e.g., layers comprised of albumin or heparin with or without other biocompatible material of synthetic or natural origin such as dextrans, cyclodextrins, polyethylene oxide, polyvinyl pyrrolidone, etc.), layers which facilitate device delivery (e.g., hydrophilic polymers such as polyvinyl pyrrolidone, polyvinyl alcohol, polyalkylene glycol, acrylate-based polymer or co-polymer compositions, etc.), epoxies and drug matrix layers which adhere to the medical device and have therapeutic agents incorporated therein or thereon for subsequent release.

When used as a drug matrix release layer for localized drug delivery, the polymer coatings of the present invention comprise any material capable of adsorbing, entrapping or otherwise holding the therapeutic agent for delivery. The drug matrix material is, for example, hydrophilic, hydrophobic, and/or biodegradable and is preferably selected from the list of polymers provided above.

The release rate of therapeutic agents from drug matrix layers is controlled, for example, by variations in the polymer structure and formulation, the diffusion coefficient of the matrix, the solvent composition, the ratio of therapeutic agent to polymer, potential chemical reactions and interactions between therapeutic agent and polymer, thickness of drug adhesion layers and any barrier layers and process parameters. The coatings used in the present invention may allow for controlled release (including both long term and/or sustained release) of a coating substance (including solid agents and therapeutic agents).

The coatings provide a suitable thickness, depending on the coating material and the purposes for which the coating is applied. For example, coatings applied for localized drug delivery are typically applied to a thickness of from about 1 mm to about 30 mm, more preferably, from about 2 mm to about 20 mm. Thin coatings of about 100 Å or very thick coatings of greater than 30 mm may also be applied. It is also within the scope of the present invention to apply multiple layers of the same or different coating materials, which may perform identical or different functions (e.g., biocompatibility, controlled drug release, etc.).

Further, it is anticipated that the method of the current invention may be used in conjunction with conventional coating methods known to those of skill in the art (e.g., ion deposition (e.g., ion beam assisted deposition, ion beam, ion beam implantation, air suspension as described in U.S. Pat. No. 6,368,658, the method described in U.S. Pat. No. 6,322,847, dipping, spraying, brushing, wiping, pad printing, electrostatic liquid spraying, electrostatic powder coating, etc.), plasma treatment, grafting or deposition, chemical vapor deposition, electroplating, etc.) to coat other agents to the medical device. Multiple layers of the same or different coating materials, which may perform identical or different functions (e.g., biocompatibility, controlled drug release, etc.) may be applied using these conventional methods.

In any embodiment of the current invention, the methods of the present invention result in complete or partial coating of the medical device. Partial coating may be accomplished using masking techniques known to those of skill in the art. It is contemplated that different portions of a medical device may be coated using the methods of the current invention, either alone or in conjunction with conventional methods. Accordingly, the various coating techniques described herein may be used in conjunction with one another and are not mutually exclusive.

### Medical Devices

The coating processes described herein may be used to coat virtually any medical device. Accordingly, medical devices useful in the present invention may be include one or more metals (e.g., stainless steel, tantalum, gold, titanium, nickel-titanium alloy, cobalt alloys, etc.), polymers (e.g., polyurethane and its copolymers, silicone and its copolymers, ethylene vinyl-acetate, poly(ethylene terephthalate), thermoplastic elastomer, polyvinyl chloride, polyolefins, cellulosics, polyamides, polyesters, polysulfones, polytetrafluoroethylenes, acrylonitrile butadiene styrene copolymers, acrylics, polyactic acid, polyclycolic acid, polycaprolactone, polyacetal, poly(lactic acid), polylactic acid-polyethylene oxide copolymers, polycarbonate cellulose, collagen, chitinfs, etc.) ceramics (e.g., aluminum ceramics, glass ceramics, etc.), composites and/or mixtures thereof.

Medical devices within the scope of the present invention include, but are not limited to, catheters, implantable vascular access ports, blood storage bags, vascular stents, biliary stents, colonic stents, bronchial stents, pulmonary stents, esophageal stents, ureteral stents, aneurysm filling coils, hypodermic needles, soft tissue clips, blood tubing, central venous catheters, arterial catheters, vascular grafts, intra-aortic balloon pumps, heart valves, cardiovascular sutures, total artificial heart and ventricular assist pump, blood oxygenators, blood filters, hemodialysis units, hemoperfusion units, plasmapheresis units and hybrid artifical organs. Any surface of the above medical devices may be coated using the methods of the present invention.

Finally, it should be noted that there are alternative ways of implementing the present invention. Accordingly, the present embodiments are to be considered as illustrative and not restrictive, and the invention is not to be limited to the details given herein, but may be modified within the scope and equivalents of the appended claims. All references and patents are herein incorporated by reference in their entirety for all purposes.

What is claimed is:

1. A method for coating at least a portion of a medical device with a solid agent, the method comprising the step of electrodepositing a powder of the solid agent on at least a portion of a surface of the medical device.

2. The method of claim 1, wherein the solid agent is a therapeutic agent.

3. The method of claim 2, wherein the therapeutic agent is selected from the group consisting of sirolimus, paclitaxel, dexamethasone, estradiol, tacrolimus, nitric oxide, mycophenloc acid and trapidil.

4. The method of claim 1, wherein the solid agent is substantially insoluble in any solvent.

5. The method of claim 1, further comprising coating the medical device with another agent.

6. The method of claim 5, wherein the other agent is another solid agent.

7. The method of claim 5, wherein the other agent is selected from the group consisting of other therapeutic agents, radiopaque materials, polymeric materials, sugars, waxes, fats and combinations thereof.

8. A method for coating at least a portion of a medical device with a solid agent, the method comprising the step of electrodepositing a powder blend comprised of the solid agent and a solid, electrically chargeable filler on at least a portion of a surface of the medical device.

9. The method of claim 8, wherein the electrically chargeable filler is selected from the group consisting of therapeutic agents, radiopaque materials, radioactive materials, polymeric materials, sugars, waxes, and fats.

10. A method for coating at least a portion of a medical device with a solid agent, the method comprising the steps of:
coating at least a portion of a surface of the medical device with an adhesive agent; and
coating said surface of the medical device coated with the adhesive agent with the solid agent.

11. The method of claim 10, wherein the portion of the surface of the medical device is coated with the adhesive agent by a process selected from the group consisting of wax and paraffin.

12. A method for coating at least a portion of a medical device with a solid agent, the method comprising the steps of:
coating at least a portion of a surface of the medical device with an adhesive agent; and
coating said surface of the medical device with a powder blend comprising the solid agent and a filler.

14. The method of claim 13, wherein said surface of the medical device is coated with the powder blend by a process selected from the group consisting of dipping, spraying, brushing, wiping, pad printing, electrostatic liquid powder coating, ion beam implantation, and air suspension.

14. The method of claim 12, wherein the adhesive agent is selected from the group consisting of wax and paraffin.

15. A powder blend comprising a solid agent and a solid electrically chargeable filler.
